(19) United States
(12) Patent Application Publication
(10) Pub. No.: US 2009/0251532 A1
(43) Pub. Date: Oct. 8, 2009

(54) Electronic endoscope system

(75) Inventor: Kazunori Abe, Saitama-shi (JP)

Correspondence Address: MCGINN INTELLECTUAL PROPERTY LAW GROUP, PLLC, 8321 OLD COURTHOUSE ROAD, SUITE 200, VIENNA, VA, 22182-3817, US

(73) Assignee: FUJIFILM Corporation, Tokyo (JP)

(21) Appl. No.: 12/385,208

(22) Filed: Apr. 1, 2009

(30) Foreign Application Priority Data
Apr. 2, 2008 (JP) 096209/2008

(51) Int. Cl. A61B 1/04

(52) U.S. Cl. 348/71; 348/E09.037

(57) ABSTRACT

An electronic endoscope system, including a scope that outputs a plurality of types of color signals obtained by an imaging device and a processor for processing the signals outputted from the scope. A color conversion matrix in which each element data value is defined such that only a color involved in diagnosis is converted to desired color is stored in a memory of the scope or in a memory of the processor. The processor includes at least one multiplier that multiplies the plurality of types of color signals outputted from the scope by coefficients, each provided for each color signal, a coefficient setting means (microcomputers) for setting each element data of the color conversion matrix read out from the memory in the multiplier as the coefficients, and at least one adder that adds up signals outputted from the multiplier.

### BACKGROUND OF THE INVENTION

1. Field of the Invention

The present invention relates to an electronic endoscope system capable of performing color conversion with a simple circuit.

2. Description of the Related Art

Generally, an electronic endoscope system is supplied as a set of a plurality of different types of electronic endoscopes (hereinafter, “scope”) and a processing unit (hereinafter, “processor”) for performing processing on an image obtained by a scope and outputting the processed image to a monitor. The processor is required to output images of stable quality regardless of the model of the connected scope. It is particularly undesirable that the image color varies depending on the model of the scope, since a doctor usually diagnoses an observation region based on the difference in color of the region. With respect to this problem, the inventors of the present invention have proposed a system in U.S. Patent Application Publication No. 20060087557, in which color values obtainable by imaging observation regions and true color values of the observation regions are related and stored in a lookup table with respect to each scope model, and color replacement is performed based on the lookup table. According to the system, true colors of observation regions can be precisely reproduced on a monitor. Further, the system allows an image color to be converted to a color that facilitates diagnosis, such as differentiating the color of the observation region, by further performing color conversion using another lookup table.

Although the system disclosed in U.S. Patent Application Publication No. 20060087557 is functionally superior, memory size becomes inevitably large since the lookup table is required for each scope model. In order to process an acquired image in real time and display on a monitor, a large, high-speed memory is required, resulting in increased cost. It is, therefore, an object of the present invention to realize a color conversion function of an electronic endoscope system with a simpler and inexpensive configuration.

### SUMMARY OF THE INVENTION

An electronic endoscope system of the present invention includes a scope that outputs a plurality of types of color signals (e.g., RBG signals, YCC signals, CMYK signals or the like) obtained by an imaging device and a processor for processing the signals outputted from the scope. The system further includes the following means. The system includes a memory having stored therein a color conversion matrix in which each element data value is defined such that only a color involved in diagnosis is converted to a desired color. The color involved in diagnosis includes colors in the range from yellow-red (YR) to red-purple (RP) in terms of Munsell color system. That is, the color involved in diagnosis refers to colors normally seen when inner walls of organs, vessels, and diseases, such as redness, are observed, and excluded those which are unlikely as the colors of organs, such as blue, green, and the like. The term “desired color” refers to a color convenient to perform diagnosis, and generally refers to a color that expands the color difference between a portion and the other portion of a region. For example, color conversion of a portion of an inner wall where redness is developed to more reddish and normal portion to yellow-reddish makes the redness more distinguishable in location and size.

The electronic endoscope system further includes a multiplier that multiplies the plurality of types of color signals outputted from the scope by coefficients, each provided for each color signal, a coefficient setting means for setting each element data of the color conversion matrix stored in the memory in the multiplier as the coefficients, and an adder that adds up a plurality of types of multiplied signals outputted from the multiplier. The multiplier and adder may be provided for each of three types of color signals outputted from the scope. But where colors not involved in diagnosis are not converted, only one multiplier and one adder may be provided to generate converted R signal.

In the configuration of the electronic endoscope system described above, the memory size required is only for storing element data of the color conversion matrix. For example, where each element data of the color conversion matrix is held as one-byte data and the matrix size is 3×3, only 9 bytes per scope model are required. This allows realization of a color conversion function simply and inexpensively with far less amount of memory in comparison with a color conversion scheme in which a color conversion lookup table is held for each scope model.

Here, a configuration may be adopted in which a variable color conversion matrix in which the element data values are rewritable and a fixed color conversion matrix in which the element data values are not rewritable are stored in the memory, and the a matrix updating means for accepting input specifying an element data value and updating the element data values of the variable color conversion matrix based on the specification is further provided in the electronic endoscope system. In this configuration, a doctor may perform diagnosis under optimum conditions for the doctor by setting an element data of the variable color conversion matrix to a favorite value. In addition, where the element data values can not be adjusted properly, the setting may be returned to the default state by reading the element data of the fixed color conversion matrix.

Preferably, the color conversion matrix is stored in a memory provided in the scope. This allows the user to carry around the color conversion matrix with the scope, so that even when the scope is used by connecting to a processor which is different from the processor the user usually uses, the user is not bothered with the matrix setting work.

Further, a configuration may be adopted in which a plurality of color conversion matrices, each defined for each observation region, is stored in the memory, and the coefficient setting means accepts input specifying an observation region and sets element data of the color conversion matrix corresponding to the specified observation region in the multiplier. This invariably enables optimum color conversion by replacing the matrix used for the color conversion even if the color convenient to perform diagnosis is different depending on the observation region (type of organ).

The electronic endoscope apparatus of the present invention reduces the amount of data required to be held in advance for color conversion by performing color conversion using a color conversion matrix and further by defining each element data value such that only a color involved in diagnosis is converted to a desired color. This may provide a color conversion function sufficient for performing accurate diagnosis with a circuit size which is about one-tenth the circuit size of a color conversion scheme using a lookup table.

### BRIEF DESCRIPTION OF THE DRAWINGS

FIG. 1 is a diagram of an electronic endoscope system, illustrating the schematic configuration thereof.

FIG. 2 is a diagram of the dedicated image processing board, illustrating the detailed configuration thereof.

FIG. 3 is a diagram of the matrix conversion section, illustrating the schematic configuration thereof.

FIG. 4A illustrates a setting information storage area of the memory in a scope according to an embodiment.

FIG. 4B illustrates a setting information storage area of the memory in the processor according to an embodiment.

FIG. 4C illustrates a setting information storage area of the memory in the processor according to another embodiment.

FIG. 5 is a flowchart illustrating processing of microcomputer 32.

FIG. 6 is a flowchart illustrating processing of microcomputer 42.

### DESCRIPTION OF THE PREFERRED EMBODIMENTS

Hereinafter, an electronic endoscope system used for inspecting a digestive organ will be described as an embodiment of the present invention.

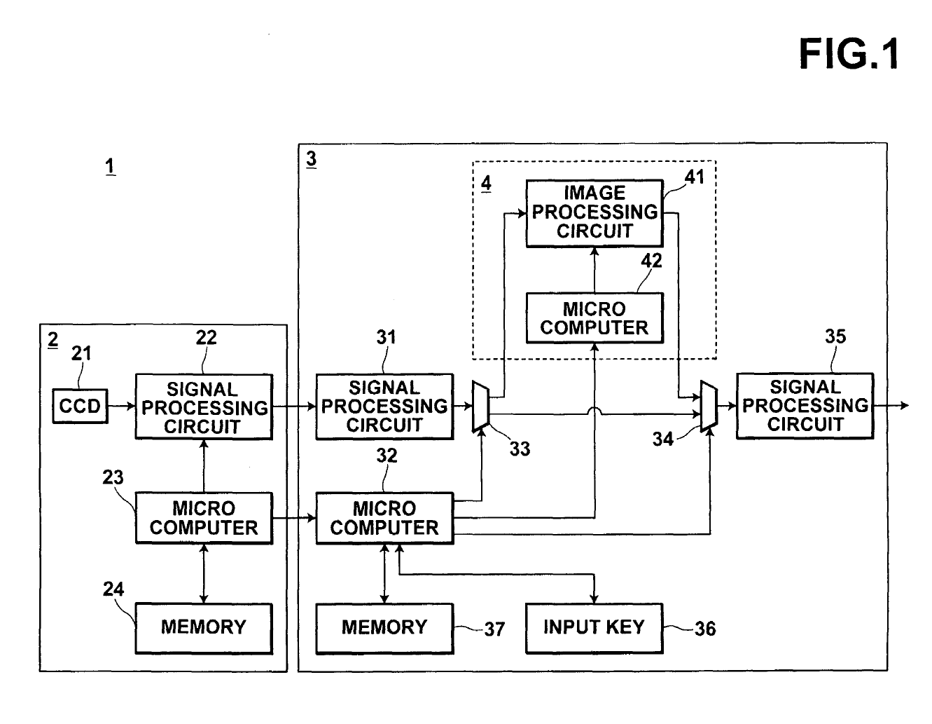

FIG. 1 is a diagram of the electronic endoscope system, illustrating the schematic configuration thereof. As illustrated in FIG. 1, electronic endoscope system 1 includes electronic endoscope 2 (hereinafter, “scope 2”), processing unit 3 (hereinafter, “processor 3”) for processing an image obtained by scope 2, a not shown light source unit, a monitor, a printer, and the like. Electronic endoscope system 1 allows the use of a plurality of different scopes according to the purpose of the inspection, scope 2 shown in FIG. 1 represents the configuration common to these scopes.

Scope 2 includes CCD (Charge Coupled Device) 21, signal processing circuit 22 for processing a signal obtained by CCD 21, microcomputer 23 for performing various controls, memory 24, and a not shown connector unit to be connected to processor 3.

CCD 21 is attached to the distal end of scope.2, together with an objective lens. CCD 21 obtains reflection light from an observation object and converts the light to electrical signals. Signal processing circuit 22 performs signal processing, such as correlated double sampling, automatic gain control, and A/D conversion, on the output signals of CCD 21. Microcomputer 23 controls the operation of the signal processing circuit and data transfer to processor 3. Memory 24 has a plurality of setting information storage areas. Each setting information storage area may store ON/OFF setting values of the functions of processor 3 and processing parameters.

Processor 3 includes a not shown connector unit. The connector unit of processor 3 has a structure that allows easy connection or disconnection of the connector unit of each scope described above.

Processor 3 includes signal processing circuit 31 that performs gamma correction on RGB signals inputted from signal processing circuit 22 via the connector unit and generates a video signal. When the output signals of signal processing circuit 22 of the scope are CMYG signals, signal processing circuit 31 also converts the CMYG signals to RGB signals. Processor 3 further includes microcomputer 32 that controls operation of signal processing circuit 31 and communication with scope 2. Signal processing circuit 35 that generates a monitor output signal by performing pixel number conversion and D/A conversion is disposed in the latter stage of signal processing circuit 31.

Processor 3 further includes memory 37 having a plurality of setting information storage areas. Memory 37 may store setting information identical to that stored in memory 24 of scope 2.

Processor 3 further includes input key 36 for inputting a character or a numerical value to microcomputer 32 from the outside. Input key 36 may be a keyboard built-in the body of processor 2 or a keyboard externally attached to processor 3.

Processor 3 further includes dedicated image processing board 4, in addition to a main board on which signal processing circuit 31, microcomputer 32, and signal processing circuit 35 are mounted. Mounted on dedicated image processing board 4 are image processing circuit 41 that performs various types of image processing on image signals outputted from signal processing circuit 31, and microcomputer 42 that controls image processing circuit 41. Image processing circuit 41 is connected to signal processing circuits 31 and 35 via selectors 33 and 34 respectively. Selectors 33 and 34 are switched based on control signals from microcomputer 32.

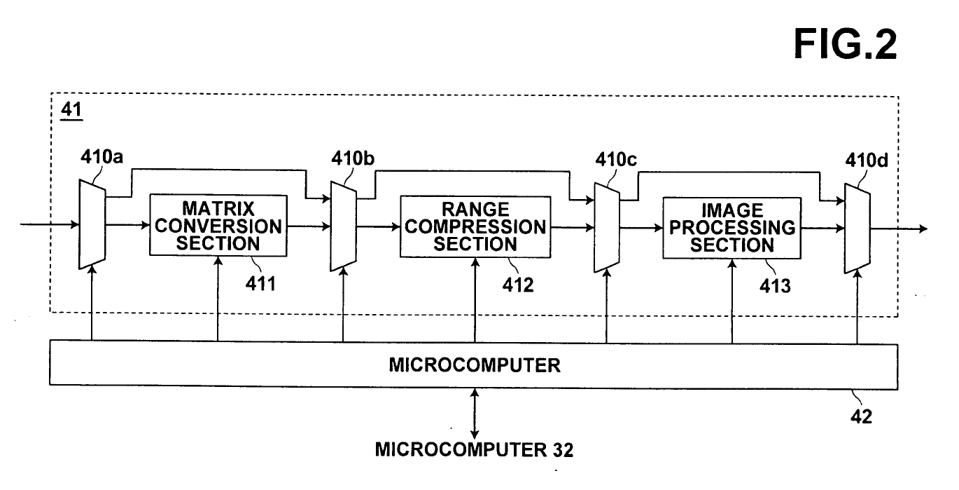

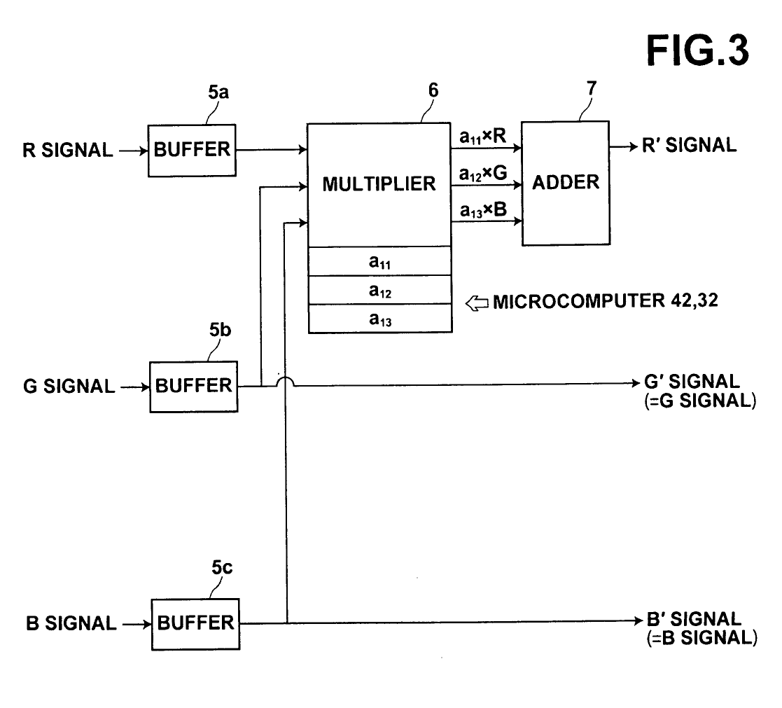

The detailed configuration of dedicated image processing board 4 is shown in FIG. 2. As shown in FIG. 3, image processing circuit 41 is divided into three processing sections: matrix conversion section 411, range compression section 412 that compresses the dynamic range of an image, and image processing section 413 that performs image processing, such as processing for improving sharpness, other than color and dynamic range conversions. Each of processing sections 411 to 413 can be selectively operated by switching selectors 410a to 410d . That is, ON/OFF of each processing function can be set individually. Selectors 410a to 410d are switched based on control signals supplied from microcomputer 42. Microcomputer 42 supplies a parameter to each processing section required for the processing.

Matrix conversion section 411 performs calculations shown in Formula (1) below on three different signals supplied from signal processing circuit 31 via selectors 33 and 410a , i.e., R signal, G signal, and B signal to generate converted signals, R′ signal, G′ signal, and B′ signal. The method for determining matrix element data all to a₃₃ will be described later.

$$[ \begin{matrix}R \\ G \\ B\end{matrix} ] = [ \begin{matrix}{a}_{11} & {a}_{12} & {a}_{13} \\ {a}_{21} & {a}_{22} & {a}_{23} \\ {a}_{31} & {a}_{32} & {a}_{33}\end{matrix} ] [ \begin{matrix}R \\ G \\ B\end{matrix} ] \qquad ( 1 )$$

FIG. 3 is a diagram of matrix conversion section 411 according to an embodiment of the present invention, illustrating the schematic configuration thereof. Matrix conversion section 411 performs calculations when matrix element data a₂₁, a₂₃, a₃₁, and a₃₂ are “0” and a₂₂ and a₃₃ are “1” in Formula (1) above, that is, it performs calculations shown in Formula (2) below.

$$[ \begin{matrix}R \\ G \\ B\end{matrix} ] = [ \begin{matrix}{a}_{11} & {a}_{12} & {a}_{13} \\ 0 & 1 & 0 \\ 0 & 0 & 1\end{matrix} ] [ \begin{matrix}R \\ G \\ B\end{matrix} ] \qquad ( 2 )$$

Matrix conversion section 411 includes three buffer circuits 5a , 5b , and 5c for tentatively storing R signal, G signal, and B signal supplied from signal processing circuit 31 via selectors 33 and 410a , a multiplier for multiplying R signal, G signal, and B signal outputted from buffer circuits 5a , 5b , and 5c by predetermined coefficients respectively, and adder 7 for adding multiplied results to generate R′ signal. The coefficients by which R signal, G signal, and B signal are multiplied by multiplier 6 are supplied from microcomputer 42 and set in multiplier 6.

FIG. 3 shows a case in which the matrix conversion section is formed with essential circuits only, but three multipliers and three adders corresponding to R signal, G signal, and B signal respectively may be provided. In this case, microcomputer 42 sets the coefficients by which the R signal, G signal, and B signal are multiplied in each multiplier.

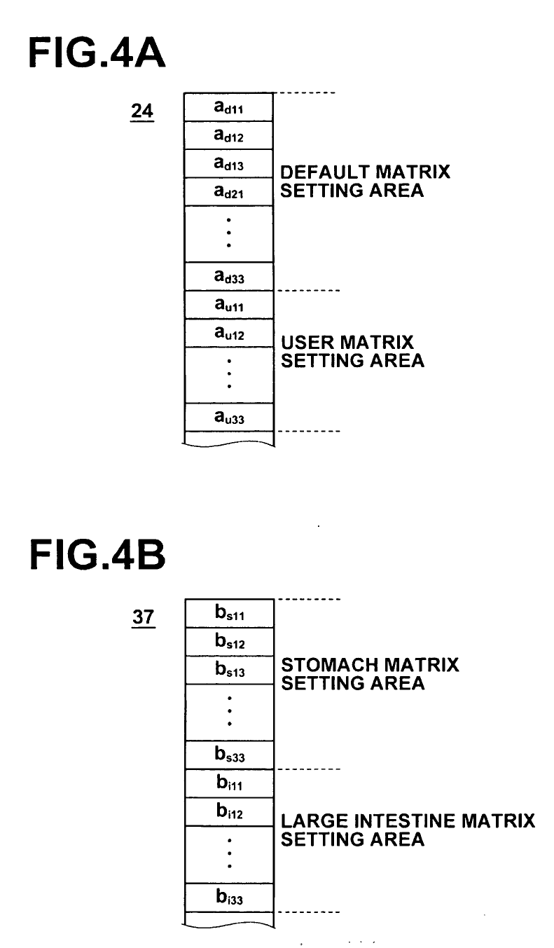

The matrix element data, i.e., coefficients set in multiplier 6 (if three multipliers are provided, to each of the multipliers) by microcomputer 42 are stored in memory 24 of scope 2 or in memory 37 of processor 3 in advance. FIG. 4A illustrates an example of matrix setting area of memory 24 in scope 2, and FIGS. 4B and 4C illustrate examples of matrix setting area of memory 37 in processor 3.

In the example shown in FIG. 4A, two areas for setting element data of two matrices are provided in memory 24, and a fixed color conversion matrix in which the element data values are not rewritable is stored in one area, as the default matrix, and a variable color conversion matrix in which the element data values are rewritable is stored in the other area, as a user matrix. Provision of two types of matrices, one of which is not rewritable and the other of which is rewritable, as in this example, allows customization according to user preferences while maintaining recommended values as the matrix element data values.

In one embodiment, the default matrix is defined such that a color obtained by a scope is converted to a color faithful to the actual color. The default matrix is defined by the manufacture and stored in the memory. Each element data value of the default matrix is determined in the following steps. First, values of colors involved in diagnosis, more specifically, those observable as the colors of inner walls of organs, vessels, redness, and the like, such as yellow-red, red, red-purple, are obtained from Macbeth 24 color chart, and color values obtained in this manner are set as the target color values of each color. Next, the colors of Macbeth chart whose target color values have been obtained are imaged by an intended scope, i.e., the scope with the memory in which a default matrix is going be stored. In this way, the color values obtainable by the scope with respect to each color are obtained. Further, a color difference between color values obtained by subjecting the color obtained by the scope to matrix conversion and the target color values is obtained with respect to each color. Then, a matrix that minimizes the total value of color differences with respect to these colors is determined as the default matrix. For the optimization of the element data, any known optimization method, such as Wiener estimation method, downhill simplex method, or the like may be used.

When obtaining a color difference, it is preferable that color values represented in RGB color system are converted to color values represented in L*a*b* color system before that. By minimizing the color difference with respect to all of the colors in L*a*b* color system, i.e., the total of each value represented by Formula (3) below, the defined default matrix becomes a matrix that allows color conversion more adapted to human vision.

\sqrt{(a*ₐᵢₘ−a*ₒᵤₜ)²+(b*ₐᵢₘ−b*ₒᵤₜ)²} (3)

(where, a*ₐᵢₘ and b*ₐᵢₘ are the target color values represented in L*a*b* color system, and a*ₒᵤₜ and b*ₒᵤₜ are the values obtained by the scope subjected to matrix conversion and represented in L*a*b* color system.)

Element data of the user matrix are set to the same values as the default matrix in the initial setting. The user may adjust each element data value of the user matrix data individually on a predetermined setting screen. For example, the user may expand the color space of an image in a predetermined range around red without changing the luminance of the entire image by increasing the value of element data all by 0.5, decreasing the value of element data a₁₂ by 0.3, and decreasing the value of element data a₁₃ by 0.2 in the matrix shown in Formula (1) above. That is, the color difference observed in the range may be enhanced. For example, where the color difference between a normal portion and a redness portion of an inner wall is small, the redness portion may become more distinguishable by expanding the color difference between them.

Now, returning to FIG. 1, an operation of the system when setting the user matrix will be described. When a setting screen is called by the operation of input key 36, microcomputer 32 in processor 3 makes a request to microcomputer 23 in scope 2 for transferring each element data value of the user matrix stored in memory 24. When each element data value of the user matrix is received, microcomputer 32 displays the received values on a predetermined display screen (now shown) and accepts a change operation for an element data value by input key 36. When a change operation is performed, the new value is tentatively stored in memory 37 and the screen display is updated. When an instruction to save the user matrix is received, microcomputer 32 transfers the new value stored in memory 37 to microcomputer 23, and instructs microcomputer 23 to update the user matrix. That is, in the present embodiment, microcomputer 32 functions as the matrix updating means.

When initialized, the user matrix is restored to the state identical to the default matrix. That is, when an initialization process is performed by the operation of input key 36 and microcomputer 23 is instructed to initialize the matrix by microcomputer 32, microcomputer 23 copies the data stored in the default matrix setting area to the user matrix setting area. If the user should have made an inappropriate adjustment, this allows resetting easily.

In the example shown in FIG. 4A, the default matrix is not necessarily a matrix that converts a color obtained by the scope to a color faithful to the actual color of an organ, and may be a matrix that performs a color conversion recommended by the manufacture.

The example shown in FIG. 4B is an example in which areas for setting element data of a plurality of matrices corresponding to observation regions, i.e., types of organs are provided in memory 37 of processor 3. FIG. 4B illustrates a case in which two areas are provided, one of which is a stomach matrix setting area and the other of which is a large intestine matrix setting area. Generally, different types of scopes are used for diagnosing stomach and large intestine, so that a color obtained by the scopes may sometimes differ. Further, color appearance desired by the user, i.e., color representation appropriate for diagnosis differs between the stomach and large intestine. Consequently, if it is allowed to define a color conversion matrix with respect to each observation region, as shown in FIG. 4B, diagnoses may always be made in the optimum environment regardless of the observation region.

The example shown in FIG. 4C is an example in which areas for setting element data of a plurality of matrices corresponding to the types of scopes are provided in memory 37 of processor 3. FIG. 4C illustrates a case in which three areas are provided for scope A, scope B, and scope C respectively. The example shown in FIG. 4C may always provide images with colors that facilitate diagnosis, as in the example shown in FIG. 4B regardless of the observation region. Further, where a plurality of different types of scopes is used for the same observation region, images with colors that facilitate diagnosis may always be obtained.

In addition, setting regions which combine the examples shown in FIGS. 4A, 4B, and 4C are also possible. For example, the default matrix setting area and user matrix setting area may be provided with respect to each scope, or they may be provided with respect to each observation region. Further, the example shown in FIG. 4A is not limited to memory 24 of a scope, and applicable to memory 37 of the processor. FIG. 3 and FIGS. 4A to 4C illustrate a case in which three types of color signals (R, G, and B) are converted by a 3×3 matrix, but in the present invention, the types of color signals are not limited to three and the size of the matrix is not limited to 3×3.

An operation of processor 3 when performing endoscopic inspection by connecting scope 2 to processor 3 will now be described. When processor 3 is powered up, microcomputer 32 performs the steps of communicating with microcomputer 23 of scope 2 to verify the connection between them. Thereafter, microcomputer 32 reads various types of setting information (including color conversion matrix) stored in memory 37 or memory 24, and controls selectors 33 and 34, as well as supplying the setting information to microcomputer 42.

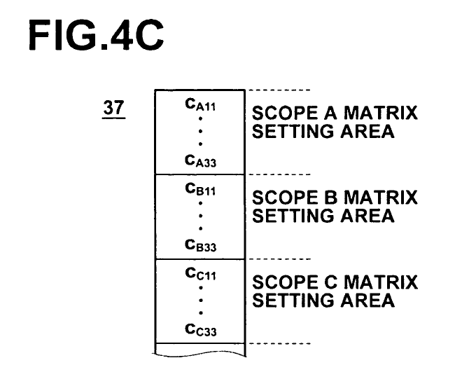

FIG. 5 is a flowchart illustrating processing performed by microcomputer 32 when setting processor 3 based on the stored setting information. In the example shown below, microcomputer 32 and microcomputer 42 function as a coefficient setting means in cooperation with each other.

When a predetermined operational procedure is performed by a user, the operational instruction is detected by microcomputer 32 (S101). If the detected instruction is an instruction to read the setting information stored in scope 2, microcomputer 32 requests microcomputer 23 to transfer the setting information stored in memory 24. Microcomputer 23 transfers the setting information read out from memory 24 to microcomputer 32. This results in that the setting information held in scope 2 is read in microcomputer 32 (S102). For example, where memory 24 has the matrix setting areas illustrated in FIG. 4A, data values stored in the user matrix setting area are read in microcomputer 32.

In the mean time, if the detected instruction is an instruction to read setting information stored in processor 3, microcomputer 32 directly accesses memory 37. This results in that the setting information held by processor 3 is read in microcomputer 32 (S103). For example, where memory 37 has the matrix setting areas illustrated in FIG. 4B or 4C, and if an instruction specifying the observation region or an instruction specifying the scope model is detected in step S101, microcomputer 32 accesses the matrix setting area of memory 37 corresponding to the specified observation region or scope model, and reads element data values of the matrix as one of setting information.

When the reading of setting information is completed, microcomputer 32 determines if it is necessary to establish connection with dedicated image processing board 4 (S104). For example, if a color conversion matrix is not stored in memory 24 or memory 37, and a compression rate of the dynamic range and a sharpness level are not set, microcomputer 32 determines that the connection with dedicated image processing board 4 is not required. On the other hand, it determines that the connection with dedicated image processing board 4 is required if any one of the setting values is set.

When a determination is made that the connection with dedicated image processing board 4 is required, microcomputer 32 controls selector 33 and selector 34 so that signal processing circuit 31 and signal processing circuit 35 are connected to dedicated image processing board 4 (S105). Then, microcomputer 32 transfers the setting information read out from memory 24 or memory 37 to microcomputer 42 of dedicated image processing board 4 (S106)

On the other hand, when a determination is made that the connection with dedicated image processing board 4 is not required, microcomputer 32 controls selector 33 and selector 34 so that signal processing circuit 31 and signal processing circuit 35 are disconnected from dedicated image processing board 4, that is, output of signal processing circuit 31 is directly inputted to signal processing circuit 35 (S107).

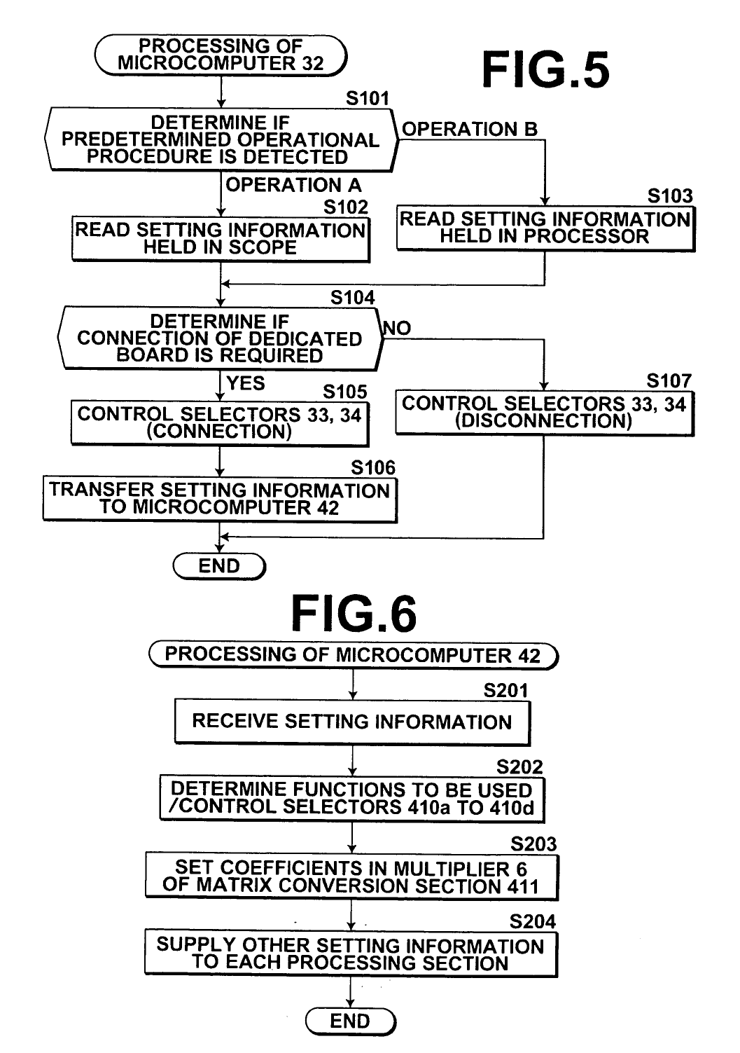

FIG. 6 is a flowchart illustrating processing performed by microcomputer 42 that receives the setting information transferred in step S106. When the setting information transferred from microcomputer 32 is received (S201), microcomputer 42 determines a function to be used, and controls selectors 410a to 410d so that only the processing section involved in the function is operated (S202). If information of color conversion matrix is received as one of the setting information, microcomputer 42 sets the received element data values in the multiplier of matrix conversion section 411 as the coefficients (S203). Further, microcomputer 42 supplies other setting information received from microcomputer 32 to each processing section (S204).

The configuration described above may largely reduce the memory usage in comparison with a color conversion scheme that uses a lookup table. For example, where each element of 3×3 matrix is one-byte data, the memory area required for storing the setting information is only 9 bytes per matrix. Even where a plurality of types of matrices is held, as illustrated in FIGS. 4A to 4C, the storage area of a dozen or few dozens of bytes is sufficient. Further, in the configuration in which only one multiplier and one adder are provided, as illustrated in FIG. 3, only a part of element data forming a matrix is required, so that the memory usage may be further reduced. Reduced memory usage allows the use of an expensive and high speed memory, whereby processing speed may be increased.

The scheme in which the setting information is held in the memory of a scope, in particular, memories for storing the setting information are required as many as the number of scope models and if a large size memory is required for that, the overall cost of the system is largely increased. In contrast, the memory usage per scope is small in the system according to the present embodiment, so that the system may provide advantageous effects of storing the setting information on the scope side, i.e., elimination of system setting each time the scope is replaced and portability of the setting information with the scope, without increasing the cost.

Further, the matrix element data are defined such that only reddish colors involved in diagnosis, such as yellow-red, red, pink, and the like, are converted to desired colors, and matrix operations by the multiplier and adder are performed only for the reddish colors, so that the circuit size and calculation amount may be reduced. Here, the observation objects of the endoscope system are limited to internal organs of humans, so that the omission or simplification of color conversions for greenish and bluish colors does not degrade the quality of an image outputted by the system.

Still further, the provision of a color conversion matrix with respect to each observation object or each scope model, and the use of an appropriate matrix by having the user to specify the observation object or the scope model allows color conversion adapted more to the diagnostic purpose or imaging environment, so that the system may output an image appropriate for the diagnosis and contribute to the improvement of diagnostic quality.

What is claimed is:

1. An electronic endoscope system, comprising
a scope that outputs a plurality of types of color signals obtained by an imaging device;
a processor for processing the signals outputted from the scope;
a memory having stored therein a color conversion matrix in which each element data value is defined such that only a color involved in diagnosis is converted to a desired color;
at least one multiplier that multiplies the plurality of types of color signals outputted from the scope by coefficients, each provided for each color signal;
a coefficient setting means for setting each element data of the color conversion matrix read out from the memory in the multiplier as the coefficients; and
at least one adder that adds up a plurality of types of multiplied signals outputted from the multiplier.

2. The electronic endoscope system as claimed in claim 1, wherein:
the memory has stored therein a variable color conversion matrix in which the element data values are rewritable and a fixed color conversion matrix in which the element data values are not rewritable; and
the system further includes a matrix updating means for accepting input specifying an element data value and updating the element data values of the variable color conversion matrix based on the specification.

3. The electronic endoscope system as claimed in claim 1, wherein the memory is provided in the scope.

4. The electronic endoscope system as claimed in claim 1, wherein:
the memory has stored therein a plurality of color conversion matrices, each defined for each observation region; and
the coefficient setting means accepts input specifying an observation region and sets element data of the color conversion matrix corresponding to the specified observation region in the multiplier.
